(19) United States
(12) Patent Application Publication
(10) Pub. No.: US 2026/0202426 A1
(43) Pub. Date: Jul. 16, 2026

(54) EARLY DIAGNOSIS SYSTEM UTILIZING STANDARD DEVIATION AND AUTOCORRELATION OF DYNAMIC FLUORESCENT OR X-RAY

(71) Applicant: Jae Won CHANG, Seoul (KR)

(72) Inventor: Jae Won CHANG, Seoul (KR)

(21) Appl. No.: 19/138,215

(22) PCT Filed: Jun. 22, 2022
(86) PCT No.: PCT/KR2022/008870

§ 371 Date: Jun. 12, 2025

(30) Foreign Application Priority Data
Jun. 25, 2021 (KR) 10-2021-0083087

(51) Int. Cl. G01N 33/68; G01N 21/64; G01N 23/20; G01N 33/575; G01N 33/58

(52) U.S. Cl. CPC G01N 33/6896; G01N 21/6428; G01N 21/6458; G01N 23/20; G01N 33/575; G01N 33/582; G01N 2021/6439; G01N 2333/4709

(57) ABSTRACT

The present disclosure relates to a method of detecting a molecule associated with the diagnosis of a disease through movement of the molecules after fluorescent labeling or by using dynamic X-rays. A method of detecting a molecule associated with the diagnosis of a disease through movement of the molecules after fluorescent labeling or labeling with a crystalline plane, according to an aspect, enables large-scale imaging and Z-axis imaging, and thus can be effectively used in the field of diagnosis.

### TECHNICAL FIELD

The present disclosure relates to an early diagnosis system using the standard deviation and autocorrelation of dynamic fluorescence or dynamic X-rays.

### BACKGROUND ART

Alzheimer's disease (AD) is the most common neurodegenerative disease in the elderly population. The major pathological characteristics of AD are the cerebral accumulation of amyloid beta (Aβ) peptides and neurofibrillary tangles (NFT) of tau, which are closely associated with neurodegenerative mechanisms that lead to toxicity and destruction of neurons and synapses during AD pathogenesis. In particular, tau is known to play a crucial role in AD pathogenesis through association with AB, and several lines of evidence have suggested a feedback loop linking tau and Aβ during tau-dependent AB toxicity and AD pathogenesis.

Tau is encoded by the microtubule-associated protein tau (MAPT) gene and exists as six isoforms in the human brain that differ in their amino-terminal inserts and microtubule-binding domain repeats. Tau serves to promote tubulin assembly and stabilize microtubule structure and function through microtubule-binding domain repeats thereof. Tau contains numerous phosphorylation sites, and the phosphorylation state of tau affects the effect thereof on microtubule assembly. Whether tau is a significant contributor to the pathogenesis of AD according to the origin of AD research is an issue currently being re-evaluated due to the disappointing results of clinical trials of AD-targeting therapeutic strategies based on the amyloid hypothesis. Positron emission tomography (PET) imaging has become a valuable technique for monitoring brain tau pathology, and a variety of recently developed tau radiotracers for the confirmation of neurofibrillary pathology through PET imaging offer great AD diagnostic and predictive potential. However, although PET imaging of tau provides rich information and reflects AD pathology quite well, PET equipment is not available in many clinical settings, and PET imaging is associated with concerns regarding high cost and radiation risk.

Alpha-synuclein (α-syn) is a highly conserved small acidic protein of 140 amino acids and a molecular weight of 19 kDa, encoded by the SNCA gene located on chromosome 4. α-syn is highly concentrated in presynaptic nerve terminals within the central nervous system, where α-syn plays a role in synaptic vesicle biology. Synucleinopathies are a set of neurodegenerative disorders associated with the deposition of fibrillar aggregates of α-syn within selective populations of neurons and glial cells. These deposits can be found as Lewy bodies (LB) in neuronal cell bodies or in dystrophic neurites in diseases such as Parkinson's disease (PD) or dementia with Lewy bodies (DLB), or in glial cytoplasmic inclusions in multiple system atrophy (MSA).

The presence of α-syn has been detected in biological fluids such as cerebrospinal fluid (CSF) and serum. Measurement of α-syn concentration in CSF by ELISA has been proposed as a biomarker for α-syn-related disorders. However, despite numerous studies showing decreased CSF α-syn levels in PD and DLB, the overall results are inconsistent. Even in those studies where reductions in CSF α-syn were demonstrated, the differences were small and there was considerable overlap within patient groups and between patients and controls. Additionally, standardization of CSF α-syn measurements between laboratories has proven to be difficult.

The aggregation properties of α-syn have recently been compared to those of prion protein, the aggregation of which causes transmissible spongiform encephalopathy (TSE). In fact, there are numerous discussions in the current literature about whether alpha-synucleinopathies are actually prion-like diseases. A recently described technique called real-time quaking induced conversion (RT-QuIC), which use the ability of prion protein to induce self-aggregation, has been used to develop a diagnostic CSF test for sporadic Creutzfeldt-Jakob disease (sCJD), the most common human form of TSE.

Therefore, the inventor of the present disclosure has completed a system that can diagnose related diseases early through the kinetic measurement of proteins with the potential for denaturation, such as amyloid beta, tau, and α-syn, using the standard deviation and autocorrelation of dynamic fluorescence or dynamic X-rays.

### DISCLOSURE OF INVENTION

### Technical Problem

An aspect is to provide a method of detecting a molecule associated with the diagnosis of a disease through movement of the molecules after fluorescent labeling, including
(i) providing a biological sample,
(ii) attaching a fluorescent label to the molecule associated with the diagnosis of a disease in the biological sample,
(iii) measuring the movement of the fluorescently labeled molecules to obtain data, and
(iv) performing regression analysis of the data.

Another aspect is to provide a method of detecting a molecule associated with the diagnosis of a disease by using dynamic X-rays, including
(i) providing a biological sample,
(ii) attaching a label to the molecule associated with the diagnosis of a disease in the biological sample,
(iii) irradiating the labeled molecules with X-rays, and measuring movement of the molecules through diffraction of the irradiated X-rays to obtain data, and (iv) performing regression analysis of the data.

### Solution to Problem

An aspect provides a method of detecting a molecule associated with the diagnosis of a disease through movement of the molecules after fluorescent labeling, including
(i) providing a biological sample,
(ii) attaching a fluorescent label to the molecule associated with the diagnosis of a disease in the biological sample,
(iii) measuring the movement of the fluorescently labeled molecules to obtain data, and
(iv) performing regression analysis of the data.

The biological sample may be a body fluid sample or a cell-based tissue sample. The biological sample may be taken from a subject for analysis using the method of the present disclosure to allow a healthcare provider to diagnose the presence or absence of a disease in a subject.

The body fluid may be selected from the group including cerebrospinal fluid, blood or blood fractions, nasal fluid or tissue, urine, feces, and lymph.

Biological samples are typically taken from a mammalian subject, preferably from a human subject. However, the biological sample may be taken from fish, bird, reptile or amphibian subjects.

A reaction sample typically includes a solvent. The solvent may be an aqueous solvent so that the reaction sample becomes an aqueous solution.

The reaction sample may be a buffered reaction sample to substantially maintain the pH of the reaction sample. For example, the reaction sample may include a biologically acceptable buffer, such as tris(hydroxymethyl)aminomethane (TRIS), phosphate buffered saline (PBS), 4-(2-hydroxyethyl)-1-piperazineethanesulfonic acid (HEPES), piperazine-N,N′-bis(2-ethanesulfonic acid) (PIPES), or Sorensen's phosphate buffer.

The reaction sample is buffered to maintain the pH of the reaction sample at about pH 6 to about pH 8.5. The reaction sample may be buffered to maintain the pH at 7.0, 7.2, 7.4, 7.6, 7.8, 8.0, 8.2, 8.4, from about 7.0 to about 8.5, or less than 9.0.

In an embodiment, the disease may be cancer or a neurodegenerative disease.

The cancer may be, but is not limited to, lung cancer, gastric cancer, liver cancer, pancreatic cancer, colorectal cancer, odontogenic carcinoma, hematologic cancer, breast cancer, brain tumor, germinoma, laryngeal cancer, esophageal cancer, bladder cancer, rectal cancer, oral cancer, uterine cancer, or gallbladder cancer.

The neurodegenerative disease includes all diseases that exhibit abnormalities in motor control ability, cognitive function, perceptual function, sensory function, and autonomic nervous system function due to decreased function or loss of neurons, and may be Alzheimer's disease, dementia, frontotemporal dementia, dementia with Lewy bodies, corticobasal degeneration, Lou Gehrig's disease, Parkinson's disease, multiple system atrophy, progressive supranuclear palsy, primary lateral sclerosis, spinal muscular atrophy, essential tremor, Huntington's disease, cerebellar degenerative disease, amyotrophic lateral sclerosis, or multiple sclerosis.

In an embodiment, the molecule associated with the diagnosis of a disease may be an organelle. Through the movement of molecules attached to organelles which are fluorescently labeled or labeled with crystalline planes, somatic cell-related diseases, including cancer may be diagnosed early or monitored.

In an embodiment, the molecule associated with the diagnosis of a disease may be a protein.

In an embodiment, the molecule associated with the diagnosis of a disease may be a denatured protein. The denatured protein may be, but is not limited to, tau protein, amyloid beta, or alpha-synuclein. The molecule associated with the diagnosis of a disease may be one or more selected from the group consisting of tau protein, amyloid beta, and alpha-synuclein.

Compared to normal proteins that undergo typical Brownian motion, rigid (stable) molecules with a relatively small radius of movement or suppressed intramolecular motion show reduced movement, whereas flexible (unstable) molecules with an increased movement radius or free intramolecular motion exhibit greater movement. There are differences in movement between native proteins and denatured proteins, and by detecting movements that vary according to the molecular radius of movement or inherent rigidity, it is possible to diagnose normal proteins, protein multimers, or pathogenic abnormal states such as denatured proteins.

In this specification, “target molecule” refers to a molecule to be detected and may refer to a molecule associated with the diagnosis of a disease.

In an embodiment, the measurement of the movement of the molecules to obtain data may be performed on a substrate.

In case that dynamic fluorescence is used, conventional materials for medical and biological experiments, such as polystyrene or polymethyl methacrylate, may be used as the substrates, but the present disclosure is not limited thereto.

For polystyrene, coating with cross-linking agents used in biological fields, such as Poly-L-Lysine, or hydrophilic treatment (plasma treatment or the like) is required. By coating an amyloid beta antibody onto a substrate that has undergone cross-linking agent treatment or hydrophilic treatment, a substrate such as a <hydrophilized polystyrene/amyloid beta antibody> substrate or a <polystyrene/poly-L-lysine/amyloid beta antibody> substrate may be completed.

In case that dynamic X-rays are used, the substrate may be any material with durability against irradiated X-rays, such as polystyrene, a polyimide film, or gold (deposition). For example, in case that a polyimide film is used, because direct binding with a protein such as amyloid beta is difficult, gold may be coated (deposited), followed by using a cross-linking agent such as SPDP (https://www.thermofisher.com/order/catalog/product/21857) or LC-SPDP (https://www.thermofisher.com/order/catalog/product/21651) that binds to a primary amine such as lysine, or an amyloid beta antibody may first be conjugated to the substrate. In particular, in case that an amyloid beta antibody can be conjugated to a gold substrate via methionine (Met) or cysteine (Cys) among amino acids in an amyloid beta antibody, a <polyimide/gold/amyloid beta antibody> substrate may be produced in the stated order without a separate cross-linking agent, and a simplified substrate targeting a specific amino acid in amyloid beta, such as a <polyimide/gold/SPDP> substrate, may also be used. In other embodiments, because polyimide can be directly coated with a cross-linking agent such as poly-L-lysine, <polyimide/poly-L-lysine> substrates may be produced. In other embodiments, in case that a material used in existing biological experiments, such as polystyrene, is used as the substrate, a substrate (<polystyrene/poly-L-lysine>) may be completed by coating with cross-linking agents used in biological fields, such as poly-L-lysine, or may further be subjected to conjugation with an amyloid beta antibody, to complete a substrate (<polystyrene/poly-L-lysine/amyloid beta antibody>), and corresponding to various tags such as his-tag is also possible.

A measurement kit including the substrate may use any holder or chamber, so long as a sample or a specimen is not damaged by evaporation or the like during measurement. The appearance of the measurement kit including a substrate, a packaging material, a filler material, and the like may take a closed form such as slide glass/cover glass, or may also take an open form such as a disposable hemocytometer or a pregnancy test kit.

A biological sample (plasma isolated from blood, saliva, rhinorrhea, or the like) may be added dropwise (in case of a closed-type measurement kit) or inserted (in case of an open-type measurement kit) into the measurement kit including a substrate. After incubation (reaction) under appropriate temperature and time conditions according to the characteristics of each substrate and the molecule associated with the diagnosis of a disease, such as amyloid beta, and the test purpose, a label may be attached to the molecule associated with the diagnosis of a disease.

In an embodiment, the label may be one or more selected from the group consisting of a metal crystal label, a polycrystalline plane label, and an antibody-conjugated label.

As the metal crystal label, metal crystals (gold, platinum, or the like) epitaxially grown on the crystalline plane of a water-soluble salt such as NaCl or KCl may be used. A solution of a cross-linking agent such as SPDP or LC-SPDP (for SPDP solutions, anhydrous ethanol is used as a solvent) may be allowed to react on a crystalline plane. Reaction conditions must be established according to the properties of the cross-linking agent and the solvent, and in case of SPDP or the like dissolved in anhydrous ethanol, a sealed container at low temperature and the like must be prepared to prevent the solvent from evaporating. Crystals to which the cross-linking agent is bound are soaked in a solvent for DXB measurement, and are separated from the crystalline plane of a water-soluble salt and recovered as a suspension. In this regard, ultrasonic waves or an appropriate dispersant (using a surfactant or the like as the dispersant) may be applied to promote dispersion of the crystal suspension to which the cross-linking agent is bound. The crystal suspension may be added dropwise (or inserted) into a specimen containing a target molecule or onto a substrate (container) on which the target molecule is immobilized. As in the above case, binding time, reaction temperature, acidity, and the like vary according to the properties of the cross-linking agent, and in case of using SPDP or LC-SPDP, a primary amine such as Lys in the target molecule is bound to the cross-linking agent by incubation at room temperature for about 6 hours (around pH 7), thereby enabling a label to be attached to the target molecule.

In case that the target molecule contains Met or a single Cys without disulfide bonds, the target molecule may be directly labeled with a crystalline plane having reactivity with sulfur atoms, such as gold crystals, platinum crystals, or palladium crystals (using Au—S covalent bonds or the like).

In other embodiments, not only the metal crystals as described above but also a polymer compound having a crystalline plane, such as polyethylene or latex may be used as a label, and labeling the target molecule after a label is allowed to react with an appropriate cross-linking agent is as described above.

As the polycrystalline plane label, any amorphous particles, such as gold colloids, that have a crystalline plane capable of causing diffraction may be used.

The polycrystalline plane label is used as a suspension of particles dispersed in a solvent. In case of using a commercially available gold particle suspension (sold by Sigma-Aldrich), the density of gold particles is increased through centrifugation or the like, followed by reaction with a cross-linking agent. Because the gold particle suspension uses water as a solvent, the surfaces of gold particles may be coated with a water-soluble cross-linking agent such as Sulfo-LC-SPDP (https://www.thermofisher.com/order/catalog/product/21650), and as needed, a process such as ultrasonic radiation or the addition of a dispersant may further be performed so that dispersion of the particles can be maintained. As in the case of the metal crystal label, reaction time, reaction temperature, acidity, and the like may be appropriately adjusted depending on the cross-linking agent. After reaction with the cross-linking agent, the particle suspension is added dropwise (or inserted) in a well-dispersed state into a specimen containing the target molecule or onto a substrate (container) on which the target molecule is immobilized. As in the case of the metal crystal label, conditions for a reaction between particles on which the cross-linking agent is applied and the target molecule must be adjusted depending on the properties of the cross-linking agent used. In case that Sulfo-LC-SPDP is used, a primary amine such as Lys in the target molecule binds with the cross-linking agent through incubation at room temperature for about 6 hours (around pH 7), thereby enabling a label to be attached to the target molecule.

In case that the target molecule contains Met or a single Cys without disulfide bonds, the target molecule may be directly labeled with a crystalline plane having reactivity with sulfur atoms, such as gold crystals, platinum crystals, or palladium crystals (using Au—S covalent bonds or the like).

In other embodiments, not only the metal crystals as described above but also a polymer compound having a crystalline plane, such as polyethylene or latex may be used as a label, and labeling the target molecule after a label is allowed to react with an appropriate cross-linking agent is as described above.

As the polycrystalline plane label, any amorphous particles, such as gold colloids, that have a crystalline plane capable of causing diffraction may be used.

The polycrystalline plane label is used as a suspension of particles dispersed in a solvent. In case that a commercially available gold particle suspension (sold by Sigma-Aldrich) is used, the density of gold particles is increased through centrifugation or the like, followed by reaction with a cross-linking agent. Because the gold particle suspension uses water as a solvent, the surfaces of gold particles may be coated with a water-soluble cross-linking agent such as Sulfo-LC-SPDP (https://www.thermofisher.com/order/catalog/product/21650), and as needed, a process such as ultrasonic radiation or the addition of a dispersant may further be performed so that dispersion of the particles can be maintained. As in the case of the metal crystal label, reaction time, reaction temperature, acidity, and the like may be appropriately adjusted depending on the cross-linking agent. After reaction with the cross-linking agent, the particle suspension is added dropwise (or inserted) in a well-dispersed state into a specimen containing the target molecule or onto a substrate (container) on which the target molecule is immobilized. As in the case of the metal crystal label, conditions for a reaction between particles on which the cross-linking agent is applied and the target molecule must be adjusted depending on the properties of the cross-linking agent used. In case that Sulfo-LC-SPDP is used, a primary amine such as Lys in the target molecule binds with the cross-linking agent through incubation at room temperature for about 6 hours (around pH 7), thereby enabling a label to be attached to the target molecule.

The antibody-conjugated label may refer to a label using an antibody to increase the selectivity of the label, and may refer to attachment of an antibody to a label. Attachment of an antibody to a label to measure kinetics with high sensitivity is a method that can perfectly complement existing fluorescent staining, or existing biochemical methods such as ELISA which have reduced applicability at low concentrations (ng/ml or less). Any commercially available antibody against the target molecule, whether produced by a firm or self-produced, may be equally applied. In other embodiments, according to the need, not only monoclonal antibodies against a specific isotype or specific molecule (peptide) but also polyclonal antibodies may be used. An amino acid in the antibody may be used for binding to a label, and as in the case of the metal crystal label and the polycrystalline plane label, a specific amino acid such as Lys, Cys, or Met, or the C-terminus, the N-terminus, or various tags may be used for binding.

A cross-linking agent such as Sulfo-LC-SPDP may be conjugated to an antibody, which is then conjugated to a label such as a metal crystal label or a polycrystalline plane label. The label conjugated to the antibody may be added dropwise (or inserted) into a specimen including the target molecule immobilized on a substrate (container) or onto the target molecule, thereby causing a reaction to occur therebetween. In case that measurement of a biological tissue-derived specimen is performed, a cross-linking agent that can allow labeling under physiological conditions may be selected.

The incubation temperature of the substrate for allowing the target molecule to be immobilized thereon and the molecule associated with the diagnosis of a disease, such as amyloid beta, may be adjusted from 4° C. to 37° C. depending on the characteristics of the substrate and the target molecule, but a reaction at 37° C. is preferable.

In case that amyloid beta is aggregated on the substrate, the reaction may require tens of hours or longer, but in the case of simple immobilization on the substrate, this is possible within a few hours.

As a label for dynamic X-rays, particles having a size of about 100 nm that can cause diffraction, such as gold crystals, gold colloids, or polymer crystals, may be used. In case that dynamic fluorescence is used as a probe, a combination of an amyloid beta antibody and a fluorescent material may be used as a label.

The principle of the detection method is to measure the Brownian motion inside and outside the target molecule, and to detect the molecule associated with the diagnosis of a disease by determining the presence or absence of abnormalities in the target molecule through changes in this motion. Referring to FIGS. 1 and 2, in FIG. 1, yellow pixels in grid lines represent the movement of fluorescently labeled molecules. Due to Brownian motion, the positions of the fluorescently labeled molecules change slightly in each frame during image capture. For each pixel, changes in fluorescence intensity are measured during frames at regular intervals to obtain a (standard deviation/mean) 2 value, and individual boxes in the boxplot of FIG. 2 represent values corresponding to 25% to 75% of the (standard deviation/mean) 2 values for all pixels. FIG. 2 illustrates a collection of values obtained using different frame intervals. Regression analysis may be performed by using the characteristics of a boxplot that emphasizes the median.

In the boxplot, the initial large error range may be due to spots newly appearing in the background. In case that the error range decreases and converges, the target molecule may be in a state bound to the cell membrane or substrate.

The movement of the individual pixels on the XY plane means movement to adjacent pixels, and the movement of the individual pixels on the Z-axis refers to an intensity reduction due to underwater scattering, refraction, or the like. Because the standard deviation of intensity changes is tracked, correspondence to movements on all the XYZ axes is possible.

For each pixel, changes in the fluorescence intensity of the pixel are measured during frames at regular intervals to calculate an autocorrelation function for each pixel. Through regression analysis of the autocorrelation functions, decay constants are calculated for each individual pixel and statistically processed. Regression analysis is performed automatically using PYTHON or C, and statistical processing is performed only for statistically and physically valid pixels by using boxplots, histograms, scatter plots, and the like. Although the median is typically used as a representative value, in some embodiments, other values such as a mean other than the median may also be set as a representative value.

In calculating decay constants using autocorrelation, the decay constants may be collected at different frame intervals in the same manner as in FIG. 2, and regression analysis may also be performed by connecting the medians of each frame interval.

Another aspect provides a method of detecting a molecule associated with the diagnosis of a disease by using dynamic X-rays, including:
(i) providing a biological sample;
(ii) attaching a label to the molecule associated with the diagnosis of a disease in the biological sample;
(iii) irradiating the labeled molecule with X-rays, and measuring movement of the molecules through diffraction of the irradiated X-rays to obtain data; and
(iv) performing regression analysis of the data.

In this regard, the biological sample and the principle of the detection method are as described above, except that dynamic X-rays are used instead of dynamic fluorescence.

In this specification, “dynamic X-rays” may be defined to include all of diffraction X-rays, transmission (reflection) X-rays, and X-rays for measuring the state of materials.

In an embodiment, the dynamic X-rays may be measured through a diffracted X-ray blinking (DXB) method. In case that a label having a crystalline plane (any particle that causes X-ray diffraction, such as gold crystals, gold particles, or polymer crystals, may be used as a label) is attached to the target molecule (in a state immobilized on the substrate) whose movement (Brownian motion) is to be observed, the label moves in synchronization with the movement of the target molecule. In this regard, in the case of being irradiated with characteristic X-rays such as Cu-Kα or X-rays filtered by a monochromator, diffraction occurs at the crystalline plane of the surface of the label, and diffraction images of the crystals may be acquired by continuous capturing. Diffraction according to Bragg conditions appears and disappears on a diffraction ring depending on the Miller index of a gold surface, appearing as if blinking. The intrinsic molecular motion may be determined through pixel-wise time-series analysis of the blinking in the diffraction images. In other embodiments, because various detection methods such as holography and absorption as well as diffraction by dynamic X-rays may be used, measurement (detection) and diagnosis using various labels corresponding thereto are possible. The time-series analysis may use autocorrelation functions, standard deviation, or the like.

Hereinafter, an analysis method using standard deviation will be described in detail.

For the consecutive values per frame of individual pixels in a fluorescently labeled area or within diffraction rings of a label in reciprocal space, changes in fluorescence intensity (photon count) or X-ray intensity (photon count) are measured during frames at regular intervals (elapsed time, Δtime) to obtain a (standard deviation/mean)² value, and then a boxplot for the corresponding elapsed time is created. Any crystal surface that causes diffraction, such as Au (111), may be used as a label.

A boxplot is created for different elapsed times, ranging from the shortest to the longest time (typically half of the measurement time) on the X-axis, but other statistical processing such as histograms or scatter plots is also possible. This method of plotting by accumulating time is not significantly different from the concept of mean square displacement (MSD) used in single particle tracking and the like. By using the characteristics of boxplots that emphasize the median, it is also possible to perform separate regression analysis by plotting the medians (in case that other statistical processing such as histograms is performed, various parameters such as peak top, mean values, and the like may be used as representative values for plotting, and statistical processing thereof is also possible).

Hereinafter, an analysis method using autocorrelation will be described in detail.

For the consecutive values per frame of individual pixels in a fluorescently labeled area or within diffraction rings of a label in reciprocal space, an autocorrelation function (ACF) is obtained as described below, and decay constants and the like are obtained through regression analysis using the negative first-order exponential function of natural logarithm.

$$ACF = 〈 I ( t ) \cdot I ( t + T ) 〉 / 〈 {I ( t )}^{2} 〉 = {y}_{0} + A \cdot {e}^{- τ \cdot t}$$

< >: time-averaged value within brackets, I(t): intensity or number of photons detected at a specific t frame, T: elapsed time (Δtime), T: decay constant, y0 and A: fitting parameters

Regression analysis is performed automatically using PYTHON or C, and unlike the analysis method using standard deviation, this method does not process all detected pixels but uses only physically and statistically significant pixels. Generally, among pixels with standard error below an appropriate value (e.g., below 20% standard error), in case that pixels have fitting parameters y0 and A greater than 0, the decay constants of the corresponding pixels are determined to be valid and subjected to statistical processing. For statistical processing of the decay constants of individual pixels, box-and-whisker plots, histograms, scatter plots, and the like are used, and usually the median is used as a representative value. In other embodiments, other values such as the mean, in addition to the median, may also be set as representative values. The larger the decay constant (fast decay of ACF), the faster the motion, and the smaller the decay constant (slow decay of ACF), the slower the motion. An analysis method such as MSD introduced in the analysis using standard deviation may also be used. Decay constants (representative values) for multiple elapsed times may be plotted for separate regression analysis, and according to the formula regarding decay constants and diffusion coefficients below, pseudo-MSD may also be obtained.

$$D = {D}_{C} {Φ}^{2} / 4 , pseudo - MSD = \int D dt$$

D: diffusion coefficient, DC: decay constant, φ: angular displacement during time constant (DC-1)

In an embodiment, the dynamic X-rays may be measured through a transmitted (reflected) X-ray blinking (TXB) method. TXB is a method that uses transmission (reflection) of X-rays, and may be the same as the DXB method, except for the use of transmitted X-rays instead of diffracted X-rays. TXB measurement has a poor s/n ratio compared to DXB, which uses diffracted X-rays, but has the advantage of being able to detect a relatively large number of photons.

### Advantageous Effects of Invention

A method of detecting a disease-related molecule through measurement of the movement of the molecules after fluorescent labeling or labeling with a crystalline plane enables large-scale imaging and Z-axis imaging, and has high sensitivity even for molecules that are rare in blood, and thus can be effectively used in the field of diagnosis.

### BRIEF DESCRIPTION OF DRAWINGS

FIG. 1 is an image showing the movement of fluorescently labeled molecules.

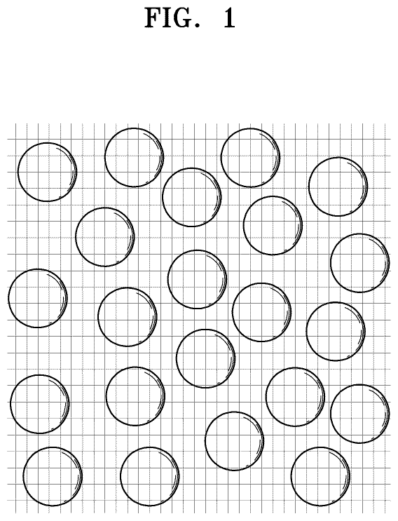

FIG. 2 is a graph showing the standard deviations of individual pixel intensities over each elapsed time (At) as a boxplot versus Δt.

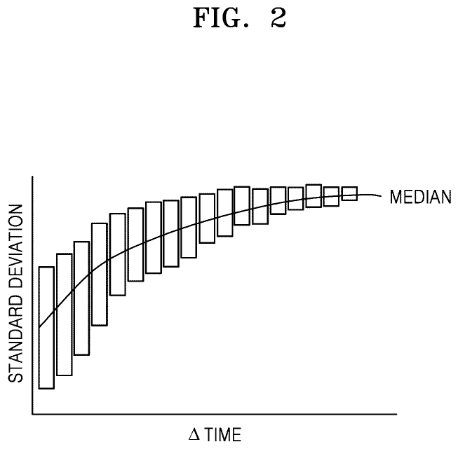

FIG. 3 illustrates the results of measuring the sizes of amyloid beta isoforms on a substrate by using dynamic light scattering.

FIG. 4 illustrates the results of detecting whether oligomerization of Aβ42 (amyloid beta-42) was inhibited, by using DXB.

FIG. 5 illustrates the results of detecting and comparing the oligomer formation of Aβ42 and Aβ38 (amyloid beta 38) (control) by using dynamic fluorescence.

### MODE FOR THE INVENTION

Hereinafter, the present disclosure will be described in more detail through examples. However, these examples are provided for illustrative purposes only and are not intended to limit the scope of the present disclosure.

The terms or words used in the specification and claims of the present disclosure should not be construed as being limited to ordinary or dictionary meanings and should be construed as meaning and concepts with the spirit of the present disclosure on the basis of a principle that an inventor can appropriately define concepts of terms to explain his/her invention in the best way.

Throughout the specification of the present disclosure, when a portion is referred to as “including” an element, unless otherwise specifically stated, this does not preclude other elements and also may mean that other elements are further included.

In the entire specification of the present disclosure, “A and/or B” means “A, or B, or A and B.”

### Experimental Example 1. Measurement of Sizes of AB Isoforms on Substrate Using Dynamic Light Scattering (DLS)

The movement of amyloid beta is closely related to the degree of aggregation of amyloid beta. Prior to measuring the mobility of amyloid beta for Alzheimer's disease diagnosis, different amyloid beta isoforms were incubated under the same conditions and the sizes thereof were measured. It was expected that isoforms with higher toxicity and aggregation degree would have larger sizes.

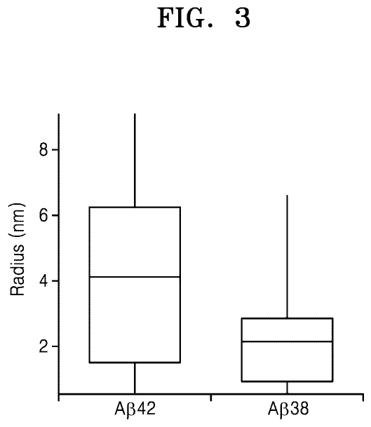

Target molecules (~1 mg/ml, PBS with Ca²⁺) were measured by DLS after incubation at 37° C. for 18 hours. The results thereof are shown in FIG. 3. As shown in FIG. 3, Aβ42 had a size (median) of 4 nm in radius, and Aβ38 had a size (median) of 2 nm in radius. Through the results, it was confirmed that Aβ42, which is known to be highly toxic and highly aggregated, was larger in size after aggregation than Aβ38.

### Experimental Example 2. Detection of Oligomerization Inhibition by Diffracted X-Ray Blinking (DXB)

To determine whether the inhibition of Aβ42 oligomerization can be detected through DXB, decay constants were measured through DXB using two different substrates.

Aβ42 is known to specifically aggregate through the C-terminal region (amino acids 41 and 42) in a form that cannot be observed in other isoforms. Therefore, by binding Met 35 and Pd (palladium) near the C-terminal domain, a reducing environment and steric hindrance were provided to the C-terminal domain, thereby creating a condition that inhibits Aβ42 oligomerization. Specifically, to prevent the C-terminal domain from inducing Aβ42 oligomerization, a substrate deposited with Pd/Cr was used as a substrate that inhibits oligomerization.

For the substrate in which Aβ42 oligomerization was not inhibited, a gold substrate coated with SPDP was used. SPDP series are widely used as cross-linking agents between gold surfaces and primary amines.

Aβ42 was loaded onto each substrate (at most 1 mg/ml, PBS with Ca²⁺). After loading, immobilization and oligomerization (aggregation) were performed by incubation at 37° C. for 18 hours.

After gold nanocrystal labeling, X-rays with a wavelength of 1.54 Å (Cu Kα, MicroMax-007 HF, RIGAKU) were incident, and the diffracted photons were detected by a PILATUS 200K array (DECTRIS) installed 30 mm away from a sample holder. Decay constants (s⁻¹) were calculated from the detected results.

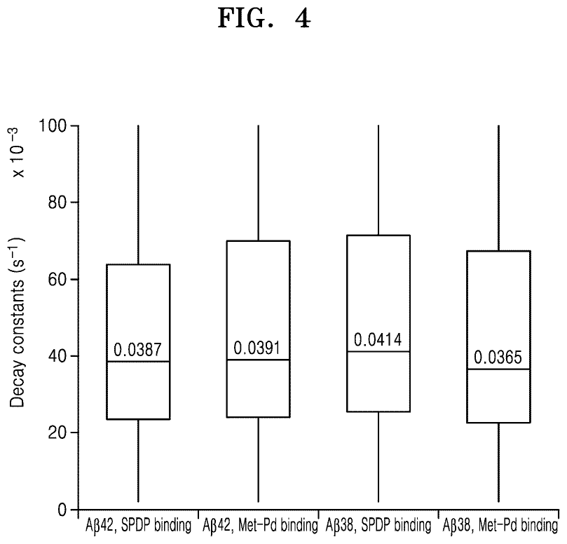

Smaller decay constants associated with Aβ that has undergone oligomerization or is in a stable molecular state indicate lower mobility, while larger decay constants associated with Aβ that is in an unstable molecular state or exists as monomers indicate faster mobility. In other words, larger decay constants indicate faster mobility and represent states with less aggregation or states that are difficult to aggregate. The results thereof are shown in FIG. 4.

As shown in FIG. 4, in case that Aβ42 oligomerization was inhibited by the binding of Met and Pd at 37° C., the average decay constant showed a larger value of 0.0396 compared to 0.0387 in case that oligomerization was not inhibited.

Aβ38 was introduced as a control, and the same measurement was performed. Because Aβ38, which is known to have lower aggregation than Aβ42, does not possess amino acids 41 and 42, it was expected that there would be no inhibition of aggregation by Met-Pd binding. Under SPDP binding conditions (Aβ38, SPDP binding), the decay constant was 0.0414, showing that the movement of Aβ38 was relatively active. This reproduces previous research findings that Aβ38 forms oligomers less than Aβ42 and the DLS results of Experimental Example 1. In contrast, in a condition that inhibits oligomerization derived from amino acids 41 and 42 (Aβ38, Met-Pd binding), the decay constant was 0.0365, which is contrary to the results from Aβ42 where oligomerization was inhibited.

Through this, it was confirmed that the inhibition or non-inhibition of Aβ42 oligomerization can be detected through DXB. This means that, by measuring the mobility of Aβ42 using decay constants through DXB, the degree of oligomerization can be detected and used for Alzheimer's disease diagnosis.

### Experimental Example 3. Detection of Oligomerization Through Dynamic Fluorescence (Dynamic FL)

To determine whether the oligomerization of Aβ42 can be detected through dynamic FL, decay constants were measured through dynamic FL.

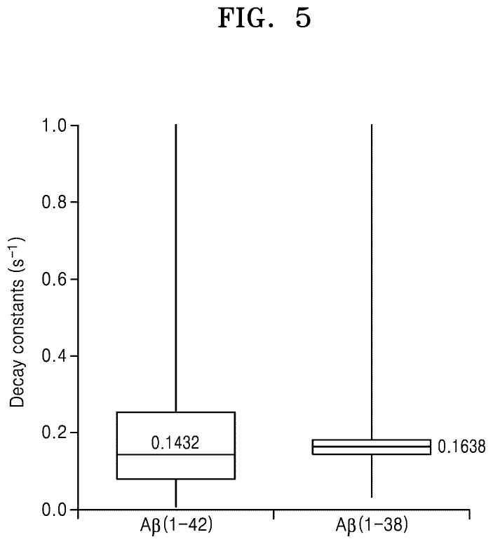

An Aβ42 solution (~1 mg/ml, PBS with Ca²⁺) was placed in an E-tube, and recovered after aggregation for 18 hours and 96 hours, and added dropwise onto culture dishes pre-coated with poly-L-lysine as a cross-linking agent. After a reaction to immobilize on the substrate (container) for 2 hours or longer, fluorescent labeling (beta Amyloid Antibody (MOAB-2) [Alexa Fluor® 350] or the like) was performed. The labeled Aβ42 was continuously photographed under a fluorescence microscope, and autocorrelation functions per pixel for the fluorescent label and the medians of the decay constants were plotted and a boxplot was drawn. The results thereof are shown in FIG. 5.

As shown in FIG. 5, the median decay constant of oligomerized Aβ42 was 0.1432, smaller than 0.1638 of Aβ38, which was less aggregated under the same conditions. Through this, it was confirmed that the oligomerization of Aβ42 can be detected through dynamic FL. This indicates that not only fluorescence correlation spectroscopy using a conventional confocal microscope, but also dynamic FL can detect the degree of oligomerization by measuring the mobility of Aβ42 as a decay constant. This also indicates that dynamic FL using a general fluorescence microscope as a measuring device can be used as an Alzheimer's disease diagnosis technology that improves the narrow measurement range of a confocal microscope.

1. A method of detecting a molecule associated with the diagnosis of a disease through movement of molecules after fluorescent labeling, the method comprising:
(i) providing a biological sample;
(ii) attaching a fluorescent label to the molecules associated with the diagnosis of a disease in the biological sample,
(iii) measuring the movement of the fluorescently labeled molecules to obtain data, and
(iv) performing regression analysis of the data.

2. The method of claim 1, wherein
the disease is cancer or a neurodegenerative disease.

3. The method of claim 1, wherein
the molecule associated with the diagnosis of a disease is one or more selected from the group consisting of tau protein, amyloid beta, and alpha-synuclein.

4. The method of claim 1, wherein
the label is an antibody-conjugated label.

5. A method of detecting a molecule associated with the diagnosis of a disease by using dynamic X-rays, the method comprising:
(i) providing a biological sample;
(ii) attaching a label to the molecule associated with the diagnosis of a disease in the biological sample;
(iii) irradiating the labeled molecule with X-rays, and measuring movement of the molecules through diffraction of the irradiated X-rays to obtain data; and
(iv) performing regression analysis of the data.

6. The method of claim 5, wherein
the disease is cancer or a neurodegenerative disease.

7. The method of claim 5, wherein
the molecule associated with the diagnosis of a disease is one or more selected from the group consisting of tau protein, amyloid beta, and alpha-synuclein.

8. The method of claim 5, wherein
the label is one or more selected from the group consisting of a metal crystal label, a polycrystalline plane label, and an antibody-conjugated label.
